(19) United States
(12) Patent Application Publication
(10) Pub. No.: US 2005/0033308 A1
(43) Pub. Date: Feb. 10, 2005

(54) Intraocular lens storage and insertion device and method of use thereof

(76) Inventors: Wayne B. Callahan, Abingdon, VA (US); Jeffery S. Callahan, Blountville, TN (US); James J. Simms, Medford Lakes, NJ (US); Dimitrii D. Dementiev, Arese (IT); William Bernard Wright, Antioch, TN (US)

Correspondence Address: WADDEY & PATTERSON, 414 UNION STREET, SUITE 2020, BANK OF AMERICA PLAZA, NASHVILLE, TN, 37219, US

(21) Appl. No.: 10/819,721

(22) Filed: Apr. 7, 2004

Related U.S. Application Data
(60) Provisional application No. 60/461,994, filed on Apr. 11, 2003.

(51) Int. Cl. A61F 9/013

(52) U.S. Cl. 606/107

(57) ABSTRACT

The present invention is a device for rolling, storing and inserting into an eye an extremely thin intraocular lens (IOL). The device performs as a roller and injector. Also disclosed herein are methods of using the lens rolling device. The chamber for rolling the intraocular lens includes curved walls, a hollow chamber, and a funnel for receiving a plunger, and a port for extruding a rolled lens. The IOL is effectively rolled by engaging the two parts of the rolling device. After rolling, the lens is ejected from the device through a cannula and into an eye.

This United States Utility Patent Application claims benefit of co-pending U.S. Provisional Patent Application Ser. No. 60/461,994 filed Apr. 11, 2003, entitled “Intraocular Lens Storage and Insertion Device and Method of Use Thereof”, which is hereby incorporated by reference.

Be it known that we, Wayne B. Callahan, a citizen of the United States, residing at 18952 Middle Dr., Abingdon, Va. 24211; Jeffery S. Callahan, a citizen of the United states, residing at 104 Eagleview Private Dr., Blountville, Tenn. 37617; James J. Simms, a citizen of the United States, residing at 37 Cochese Circle, Medford Lakes, N.J. 08055; Dimitrii D. Dementiev, M. D., a citizen of Italy, residing at Via Campo Gallo 21/25, Arese (MI) 20020, Italy; and William Bernard Wright, a citizen of the United States, residing at 556 Cedarmont Drive, Antioch, Tenn. 37013, have invented a new and useful “Intraocular Lens Storage and Insertion Device and Method of Use Thereof.”

### BACKGROUND OF THE INVENTION

Intraocular lenses (IOLs) were developed a number of years ago to replace any clouded natural lens, called a cataract. Cataracts cause individuals to lose their sight, either partially or completely, because clouding prevents light and an image from being transmitted through the lens onto the retina. When the clouding becomes severe, an individual can no longer see. Replacement of the natural lens with an IOL has become an accepted procedure for alleviating the symptoms of a cataract.

Various surgical procedures have been developed for removing a cataract, ranging from physically lifting the lens from the membrane that encapsulates the lens to emulsifying the lens through the use of sound waves and suction equipment. It has been found that this latter procedure, known as phacoemulsification, is advantageous because a much smaller incision is required in the eye, generally 3 millimeters (mm) or smaller.

A smaller incision is desirable because sutures are generally not required and the incision heals itself. If sutures are used to close a larger incision, typically up to 6.5-8 mm, the eyeball is deformed. Further, with incisions under 3 mm, the lack of sutures offers an even further assurance that the ocular globe or eyeball will not be deformed.

A number of different attempts have been made to develop IOLs which can be inserted through the smaller incision openings. Before the availability of IOLs formed of a soft material that could be deformed or compressed, various techniques were attempted to develop a small profile IOL, ranging from forming lenses with a narrower lateral dimension to various types of lenses that could be dismantled or manipulated and rebuilt in the eye.

After IOLs formed of silicon or a hydrogel material became available, IOLs could be folded, rolled or otherwise deformed or compressed so that they could be inserted into the eye through a much smaller incision than previously possible. Such lenses are described in U.S. Pat. No. 4,573,998 to Mazzocco.

Various techniques and equipment have been developed for folding soft IOLs and inserting them into the eye. These include the use of forceps with relatively long blades which can engage an IOL and hold it in a folded position while it is inserted into the eye. Such technique is shown in U.S. Pat. Nos. 5,007,913; 5,100,410 and 5,178,622. The disadvantage of these forceps devices is that they are difficult to operate. For example, as the forcep blades release the IOL, the positioning of the IOL is not tightly controlled within the eye. Further, movement of the forcep blades could cause the incision to be enlarged. Any movement close to the inner surface of the cornea is undesirable because the forcep blades or lens could rub against the endothelial cells on the inner surface of the cornea, which are not regenerative, and cause permanent damage. Since the forceps are manually squeezed by the surgeon, there is also the possibility that too much pressure could damage various portions of the IOL.

For example, a number of IOL inserters have been developed where an envelope or paddle is moved to project from the distal tip of the inserter. Such IOL inserters operate to fold the IOL as it is pulled back into the inserter. The IOL is implanted when the paddle is then moved to project from the tip. See, for example, U.S. Pat. Nos. 4,836,201; 4,880,000; 4,934,363 and 5,098,439.

Other IOL inserters have jaw-like portions that operate to fold the IOL as they close or telescopic sections that move relative to each other to hold the lens after it has been folded. See, for example, U.S. Pat. Nos. 4,714,373; 4,747,404 and 4,834,094.

An inserter was also developed, as shown in U.S. Pat. No. 4,919,130, where a cannula was designed to receive an IOL that is partially folded. A first plunger pushes the IOL through a rigid chamber of gradually diminishing diameter to fold it completely. A second plunger then pushes the IOL out of the cannula and into the eye.

In another inserter, shown in U.S. Pat. No. 4,681,102, an IOL is placed in an open cartridge which has two tabs or wing-like sections that are hinged together. The IOL is folded as the sections are closed.

Because of the moving parts in many of the folding devices discussed above, the IOL can easily be pinched or torn during the folding or insertion process.

In addition, folding and loading an IOL requires a certain amount of manual manipulation of the IOL, which takes time and complicates the surgical procedure. For example, in the device where a cannula is used, a first plunger is used to fold the lens, which must be removed and replaced by a second plunger for inserting the lens in the eye.

Thus, there is a need for an apparatus and method for rolling and storing an IOL and positioning it for insertion in the eye which removes the disadvantages of the currently available devices and methods.

### SUMMARY OF THE INVENTION

The present invention provides an intraocular lens rolling, storing and insertion device which eliminates the disadvantages associated with the currently known designs.

The lens rolling system includes a first member having a first concave surface, and a second member having an opening defined by a first shelf, a second shelf, and a second concave surface, wherein the first member removably engages the opening of the second member, so that a cavity is formed. The lens rolling system may also include the second member having an operational cannula, wherein the operational cannula aligns with the cavity so that a lens located in the cavity may exit the cavity through the operational cannula. Also, the lens rolling system may include the second member having an operational funnel, wherein the operational funnel aligns with the cavity so that the operational cannula, the cavity, and operational funnel define an opening therethrough. It also includes a rod removably engaged to the operational funnel, cavity, and operational cannula. The lens rolling system further includes a luer lock attached to the second member.

The present invention also discloses a method of rolling and inserting an intraocular lens including providing a first member having a first concave surface; providing a second member having an opening defined by a first shelf, a second shelf, and a second concave surface; inserting a lens into the opening of the second member; engaging the first member with the second member so that the lens rolls upon itself; and moving the lens from the engaged first member and second member into an eye. The method also includes pushing the lens through an operational cannula. The method further includes attaching a syringe to the second member and irrigating the lens with a fluid. In certain embodiments, moving the lens further includes contacting the lens with a rod and advancing the lens through the operational cannula and out of the engaged first member and second member. The method also includes placing a tip of the operational cannula into an incision of the eye.

The present invention discloses an apparatus for rolling, storing, and inserting an intraocular lens, including a first member having a first arm, a first cannula half, a first funnel half, a first surface, a second surface, and the first member having a first hole defined therein; and a second member having a second arm, a second cannula half, a second funnel half, a third surface, a fourth surface, and the second member having a second hole defined therein. The apparatus also includes a plunger slidably engaged with the first funnel half and the second funnel half.

Regarding the present invention, the method of rolling an intraocular lens includes providing a lens rolling device, having a first member and a second member; placing a lens on a shelf of the first member; and sliding the first member into engagement with the second member so that the lens is rolled and compressed within a cavity formed by the engagement of the first member and second member. The method also includes positioning a plunger in the cavity; and pushing the plunger through the cavity so that the lens is expelled from the cavity. In certain embodiments, placing the lens on the shelf includes placing the lens between two shelves. The method includes inserting the lens into an eye through an incision in a cornea. The incision in the cornea is from about 2 millimeters to about 0.5 millimeter.

The lens rolling device includes a first member having a first insertion arm, a first shelf, a first concave surface, the first concave surface having a first end and a second end, and the first member having a first hole defined therein; and a second member having a second insertion arm, a second shelf, a second concave surface, the second concave surface having a first end and a second end, and the second member having a second hole defined therein, wherein a cavity is formed when the first concave surface and the second concave surface engage. The cavity may be round, oval, or any combination thereof. The device includes a first cannula half attached to the first end of the first concave surface and a second cannula half attached to the first end of the second concave surface of the second member, wherein the first cannula half has a first distal end and the second cannula half has a second distal end, wherein a diameter of the first distal end of the first cannula half and a diameter of the second distal end of the second cannula half are about 1.32 millimeters. Further, the length of the first distal end of the first cannula half and a length of the second distal end of the second cannula half are about a thickness of a cornea. The device includes a first funnel half attached to the second end of the first concave surface and a second funnel half attached to the second end of the second concave surface so that the first funnel half and the second funnel half are opposite of the first cannula half and second cannula half. A container may be provided so that the device is sealed and sterilized in the container. Finally, the device includes a plunger frictionally engaged with the cavity so that the first funnel half and the second funnel half align the plunger and the full cavity.

The lens rolling device includes a first member having an insertion arm, a shelf, a half of a roller cavity, and an opening for receiving an insertion arm; and a second member having an insertion arm, a shelf, a half of a roller cavity, and an opening for receiving an insertion arm. In certain embodiments, the lens rolling device may also include a first member having a portion of a cannula and portion of a funnel; a second member having a portion of a cannula and a portion of a funnel; and a plunger.

The present invention also discloses a method of using the storage and insertion device. The method of using the storage and insertion device (1) eliminates moving parts which can pinch or tear the IOL, (2) reliably delivers the IOL into the eye without damaging either the IOL or the eye, and (3) eliminates unneeded steps in the folding process. The method includes providing the lens rolling device which has a first member and a second member, placing a lens on the first member, sliding the first member into engagement with the second member so that the lens is rolled and compressed within a cavity which is formed by the engagement of the two members. This method may further include positioning a plunger in the cavity, and pushing the plunger through the cavity so that the lens is expelled from the cavity and into the eye of a patient. The method may also include transporting the rolled lens from the rolling device to the eye of a patient in any conventional manner.

As further described herein, the first member is placed in engagement with the second member so that the lens is rolled and compressed within a chamber that is formed by portions of the first member engaging portions of the second member. Similarly, a funnel, used to guide a plunger during expulsion of the rolled lens, is formed by the engagement of the two members. Also, a cannula, which is used to deliver the rolled lens into an eye, is formed by the engagement of the two members.

Accordingly, one aspect of the present invention is to provide a lens rolling device that rolls a lens for insertion into an eye through a small incision.

Another aspect of the present invention is a lens rolling device which provides a cannula through which a rolled lens is delivered into an incision of the eye.

Still another aspect of the present invention is a lens rolling and delivery device providing a seamless cavity through which a rolled lens travels in order to be delivered into an eye.

Still another aspect of the present invention is the formation of a rolled lens cavity, a cannula, and a funnel by the engagement of two separate members in order to form a seamless cavity through which a rolled lens is pushed by a plunger without snagging or tearing the lens.

Another aspect of the present invention is to provide a method of rolling, storing, and inserting an intraocular lens in a time efficient manner.

Still another aspect of the present invention is to provide a method of rolling and inserting an intraocular lens into an eye so that the lens is delivered into an eye by placing the tip of the cannula within the incision.

### BRIEF DESCRIPTION OF THE DRAWINGS

FIG. 1 is an elevated side view of one embodiment of the present invention. The figure shows the engagement with the first member with the second member in order to create a cavity.

FIG. 2 is a perspective view the first member of an embodiment of the present invention. The first member and the second member are assembled by aligning the arms into the corresponding holes on the opposite member. The first member has a first shelf on which a lens can be placed for the rolling procedure. Also shown are the shoulder stops which prevent over compression of the lens within the cavity.

FIG. 3 is a top view of the second member of an embodiment of the present invention. The figure shows the arm and the hole present within the member.

FIG. 4 is a side elevation of an embodiment of the present invention, along with an intraocular lens, submerged in balanced salt solution within a container.

FIG. 5 is an elevated side view of an embodiment of the present invention showing the engagement of the first member with the second member. The hatched lines show the hidden position of the arms being inserted into the holes. As engagement occurs, the lens is rolled and compressed within the cavity.

FIG. 6 is a diagram of an intraocular lens being rolled within an embodiment of the present invention. The lens is placed on the lens holding shelf. The lens is placed with the lenticular surface or continuous convex surface facing away from the lens holding shelf on which it sits, and the concave surface of the lens facing the holding shelf on which the lens sits. As the first member and the second member are engaged, the first edge of the lens starts to roll inwardly. As the cavity is formed, the lens is rolled and compressed to fit within the cavity. As the first edge of the lens starts to roll, the second edge remains in approximately the same position as prior to applying pushing forces on the first member and second member of the lens rolling device.

FIGS. 7A and 7B are perspective views of an embodiment of the present invention. In FIG. 7A, the first member is shown having a first cannula half and a first funnel half which are used to form the operational cannula and operational funnel upon engagement with the second member. The distal end of the first cannula half has a narrower wall as compared to the portion of the first cannula half which is located next to the first concave surface. In FIG. 7B, the second member is shown having a second cannula half and a second funnel half which are used to form the operational cannula and operational funnel upon engagement with the first member

FIG. 8 is a side elevation of an embodiment of the present invention. The invention has an operational cannula and operational funnel. Also shown is the distal end of the operational cannula. The distal end having a narrower wall for insertion of the distal end through the insertion in the cornea in order to deliver the rolled lens of the eye of the patient. The operational funnel is used to guide the plunger into the cavity in order to push the lens from the cavity and through the operational cannula.

FIG. 9 is an elevated cross section side view showing the placement of the lens rolling device into the injector barrel. An alignment tab is placed behind the front bulkhead to position the lens rolling device snugly between the bulkhead and the alignment tab. The top surface and bottom surface of the injector barrel are sized to keep the lens rolling device fully engaged. The operational cannula protrudes through a hole in the front bulkhead. The rib and the back plate each have holes along the center line of the injector barrel for receiving the plunger.

FIG. 10 is a side elevation of an embodiment of the present invention showing the operational cannula and operational funnel.

FIGS. 11A and 11B show an embodiment of the second member. FIG. 11A shows a top view of an embodiment of the second member with hatched lines showing the cavity therethrough. Also shown is the opening of the second member. FIG. 11B shows an elevated side view of the first member and second member with hatched lines showing the opening and cavity therethrough.

FIGS. 12A and 12B show the first member and second member positioned for engagement, and a close up of the first member. FIG. 12A provides an enlarged view of the first concave surface of the first member shown in FIG. 12B. FIG. 12B is a cross sectional view of the first member and second member drawn along line 2-2 of FIG. 11B, showing the lens positioned in the opening and the first member positioned to be inserted into the opening in order to roll the lens.

FIG. 13 is an elevated side view of the invention, showing the first member inserted into the opening of the second member and a syringe attached to the second member by the luer lock. Also shown is the rolled lens being pushed into the operational cannula by a rod, which is attached to the plunger of the syringe. Hatched lines show the cavity therethrough. Hatched lines show the position of the plunger inside of the syringe. Also shown is the rod being pushed through the cavity in order to move the rolled lens.

FIG. 14 is an elevated side view of the rod attached to the gasket and plunger. The casing of the syringe is not shown.

FIG. 15 is a view of the lens rolled within the cavity. Note that the flatter of the two concave surfaces (26) is the surface in contact with the portion of the lens that is located on the exterior portion of the rolled lens.

### DESCRIPTION OF THE PREFERRED EMBODIMENTS

The present invention relates to intraocular lenses formed of a material such as a hydrophilic acrylic, hydrophobic acrylic, silicone, co-polymer or other material that allows the intraocular lens (IOL) to be folded, rolled or otherwise deformed or compressed. The present invention is a device and method for rolling, deforming or compressing IOLs and positioning them for insertion into the eye of a patient.

Most intraocular lenses have a center thickness of approximately one millimeter or greater for a 20 diopter lens. The commonly known rolling instruments include instruments to compress the thicker lenses made of a soft, flexible material, which typically allows the thicker lenses to be implanted through an insertion having a size of four millimeters or less. Thicker lenses made of a soft, flexible material can be folded upon themselves and implanted. Such folding is shown in the teachings of Mazzocco and others.

The current invention rolls a very thin intraocular lens and is designed to allow the rolled lens to be implanted into an eye through an incision which is two millimeters or less. The lens 12 can be shipped in the lens rolling device 10, but not rolled. After the lens is rolled in the lens rolling device 10, the lens 12 and the injector roller, also called lens rolling device 10, can then be placed in a bottle, or container 14, containing a balanced salt solution, sealed and sterilized. The lens rolling device 10, also called a rolling and storage device, may be manufactured of any hard plastic, ceramic material, metal, or equivalent which is compatible with human tissue. The lens rolling device 10 may be sterilized by autoclaving. In certain embodiments, autoclaving may occur at temperatures from 121° C. to 135° C. These materials are readily commercially available and known to those of skill in the art. The material could be moldable and should be durable to function under the conditions described herein. Those of skill in the art are familiar with the standard processes of molding, or tooling, etc. which may be employed to make the invention disclosed herein.

An additional aspect of the present invention is that the lens rolling device 10 and an unrolled lens 12 are shipped within a container 14. The container 14 has a balanced salt solution, or equivalent used for short term storage, so that the lens rolling device 10 and an unrolled lens 12 are submerged. The lens rolling device 10 may be used to roll the lens 12 and the container 14 may be used to store the rolled lens 16 for a short period prior to insertion into the eye of a patient. Briefly, when the lens rolling device 10 is ready to be used, the container 14, also called a bottle, is opened to expose the previously sealed and sterilized lens rolling device 10 and unrolled lens 12. After the lens rolling device 10 rolls the lens 12, as described herein, it can be placed in a plunger assembly to allow insertion of the lens 12 into the eye of a patient.

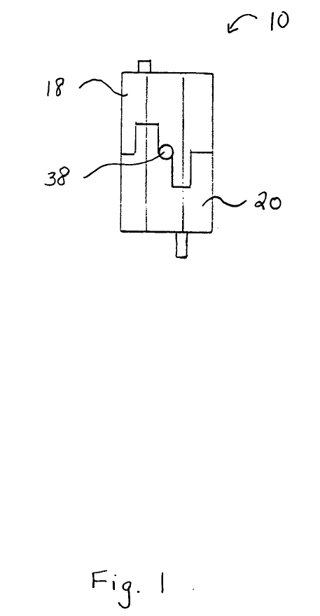

FIG. 1 shows the lens rolling device 10 in a fully assembled orientation. The lens rolling device 10 also called a rolling and storing device, or injector roller, may be manufactured of any hard plastic, ceramic material, metal, or equivalent material which is compatible with human tissue. The material should be moldable and should be durable to function under the conditions described herein. Methods of manufacturing specifically shaped hard plastic, ceramic material, metal, or equivalent material are well known in the art. The lens rolling device 10 disassembles into a first member 18 and a second member 20.

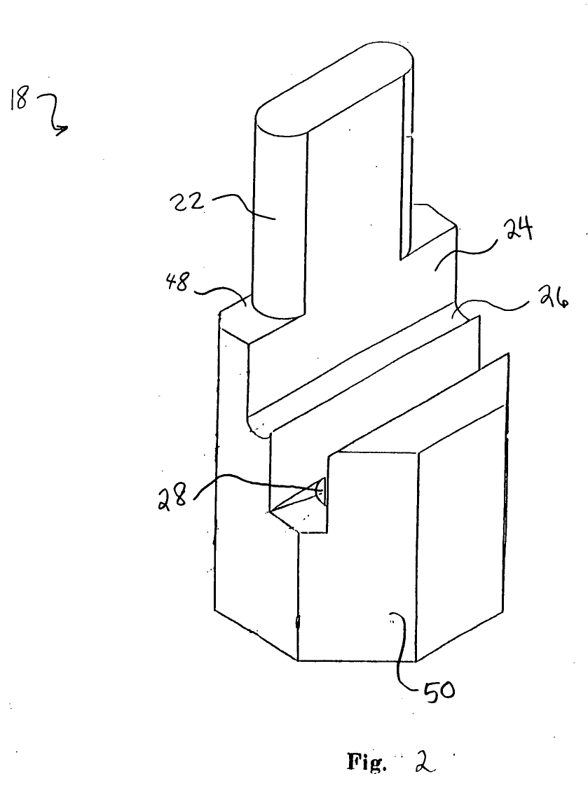

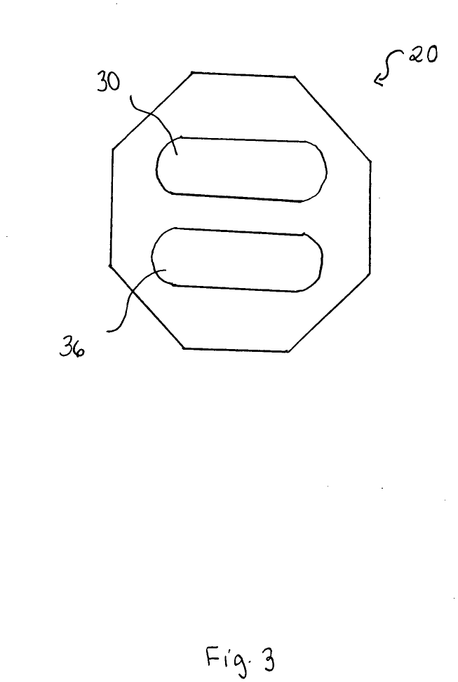

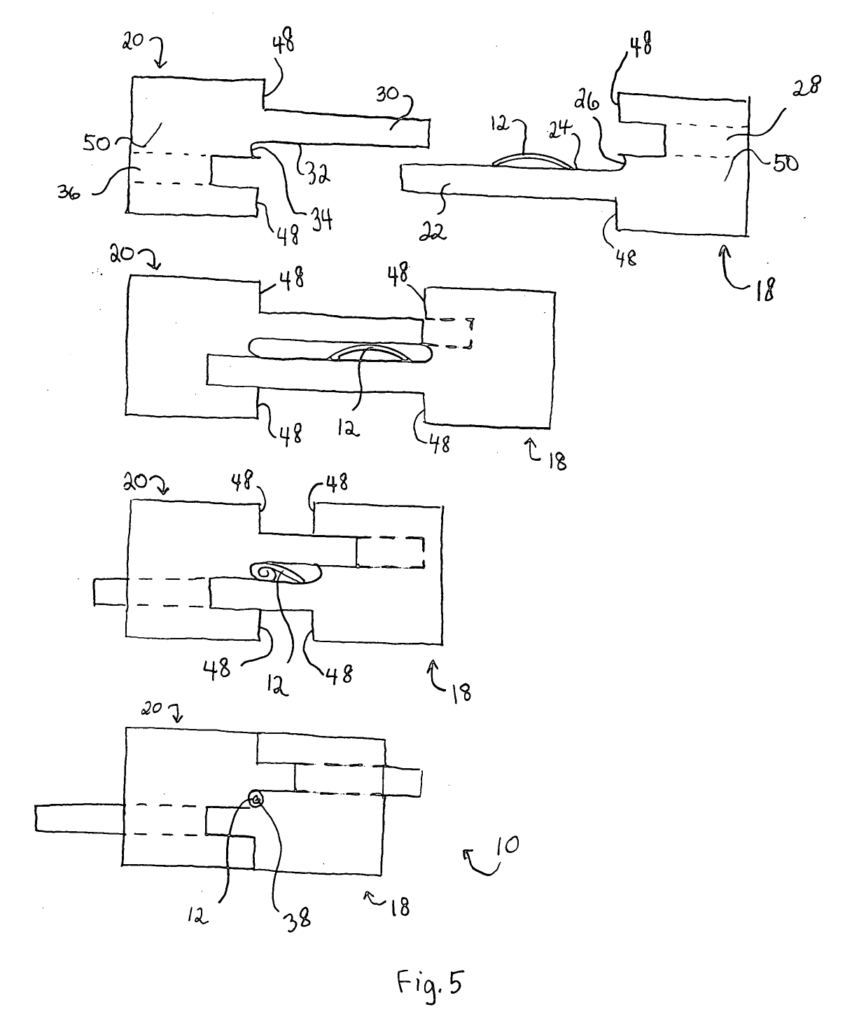

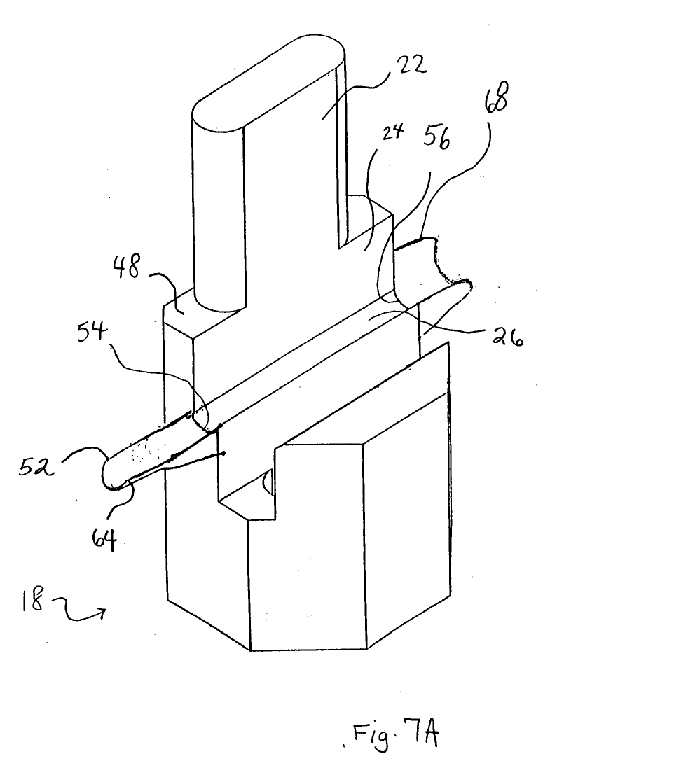

FIG. 2 shows a half of the lens rolling device 10. The first member 18 and second member 20 have similar structural features. The first member 18 includes a first insertion arm 22, also called a first arm, a first shelf 24, also called a first surface, a first concave surface 26, also called a second surface, and a first hole 28. The similar structural features for the second member 20 are shown in FIGS. 5 and 7B. The second member 20 includes a second insertion arm 30, also called a second arm, a second shelf 32, also called a third surface, a second concave surface 34, also called a fourth surface, and a second hole 36, shown in FIG. 3.

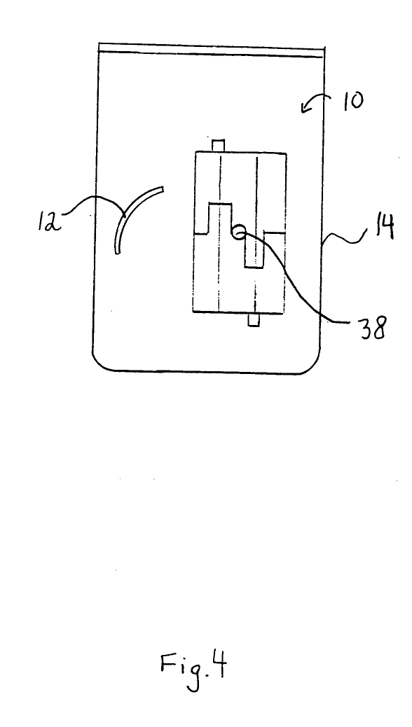

As shown in FIG. 4, the lens rolling device 10 and unrolled lens 12 may be shipped in a container 14, also called a bottle. The lens 12 is an intraocular lens formed of a material such a hydrophilic acrylic, hydrophobic acrylic, silicone, or other material that allows the intraocular lens to be folded, rolled or otherwise deformed or compressed. For illustration, but not limitation, an example of a lens 12 is disclosed in U.S. Pat. No. 6,096,077. The container 14 may be a bottle or vial capable of being sterilized and sealed, as known in the art. Containers 14 are commercially available and the method of manufacture is known to those of skill in the art. The lens rolling device 10 and lens 12 are suspended in a balanced salt solution, or equivalent within the container 14. Balanced salt solution is readily commercially available and its method of manufacture is known to those of ordinary skill in the art. The container 14, and its contents, are sterilized and sealed to be transported. When ready to use the lens 12, the lens rolling device 10, with the lens 12 in an unrolled state, are removed from the container 14. The lens rolling device 10 is used as described herein in order to roll or compress the lens 12. While the lens 12 is rolled within the lens rolling device 10, as further described below, both components may be stored in the balanced salt solution for up to thirty minutes, prior to insertion of the lens 12 into the eye of a patient.

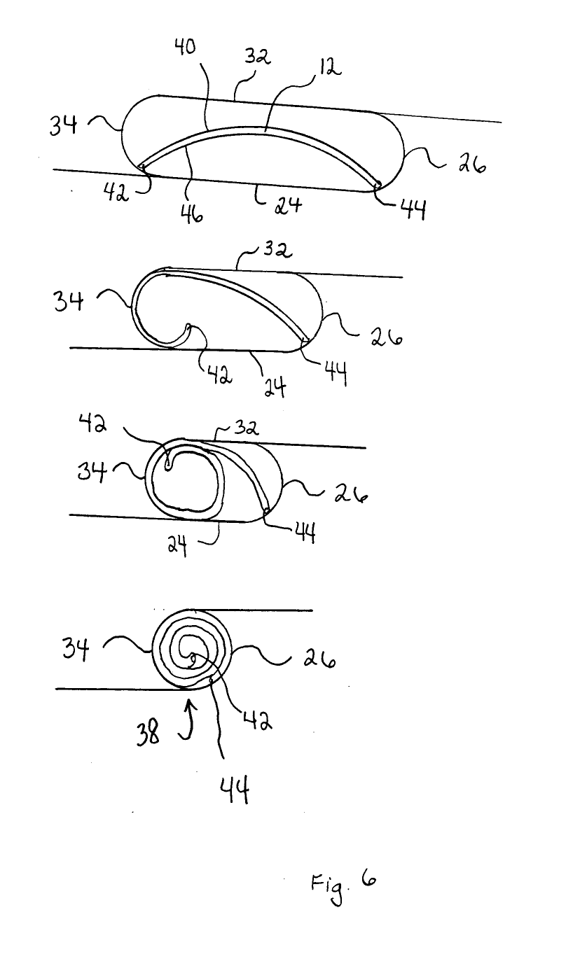

FIG. 5 shows the engagement with the first member 18 with the second member 20 which results in the assembled lens rolling device 10. The action of engagement also results in the rolling and/or compression of the lens 12. Details of the rolling and/or compressing are provided below within FIG. 6. Prior to engaging the first member 18 with the second member 20, the lens 12 is placed on the first shelf 24. In an alternate embodiment, the lens 12 may be placed on the second shelf 32 by flipping the orientation of both the first member 18 and the second member 20. The proper orientation of the first member 18 and second member 20 is shown in FIG. 5. Engagement occurs when the first insertion arm 22 is aligned with and inserted into the second hole 36 and the second insertion arm 30 is aligned with and inserted into the first hole 28. Such an orientation results in the second shelf 32 of the second member 20 being located above the lens 12. Further insertion of the arms into the holes results in the formation of a cavity 38. The cavity 38 has a diameter of approximately one millimeter. In alternate embodiments, the cavity 38 has a diameter of approximately two millimeters. In yet another alternate embodiment, the cavity 38 has a diameter from about one millimeter to two millimeters. In still another embodiment, the cavity 38 has a diameter of 0.5 millimeters to 1.0 millimeters. In certain embodiments, the cavity 38 is circular or round. In other embodiments of the present invention, the cavity 38 is oval, or a combination of circular and oval.

FIG. 6 shows the formation of the cavity 38 by the engagement of the first concave surface 26 of the first member 18 and the second concave surface 34 of the second member 20. The lens 12 is placed on the first shelf 24 of the first member 18 so that the convex surface 40 is facing away from the first shelf 24. As the first concave surface 26 moves towards the second concave surface 34 the first edge 42 of the lens 12 begins to roll. Upon continued advancement of the first concave surface 26 and second concave surface 34 the lens 12 continues to roll and shown in FIG. 6. At or near engagement of the first concave surface 26 with the second concave surface 34 the cavity 38 is formed. Within the cavity 38 is the rolled lens 12 with the first edge 42 located within the rolled lens 12 and the second edge 44 located at the exterior of the rolled lens 12. Since the lens 12 has a convex surface 40 and a concave surface 46, the lens 12 will roll towards the concave surface 46. The rolled lens 12 may be stored within the lens rolling device 10 for up to thirty minutes when submerged in a balanced salt solution or equivalent. In certain embodiments, the rolled lens 12 is submerged for approximately twenty minutes, for example, while a surgeon is extracting a cataractous natural lens of the eye. The shoulder stops 48, shown in FIG. 5, prevent overcompression of the rolled lens 12 by preventing further movement of the first member 18 towards the second member 20. Engagement of the first member 18 and second member 20 is accomplished by holding and engaging those members with one's hands along the body 50 of each member. The cavity is shown in FIGS. 1, 4, 5 and 6.

Multiple Embodiments of the Device for Rolling a Lens

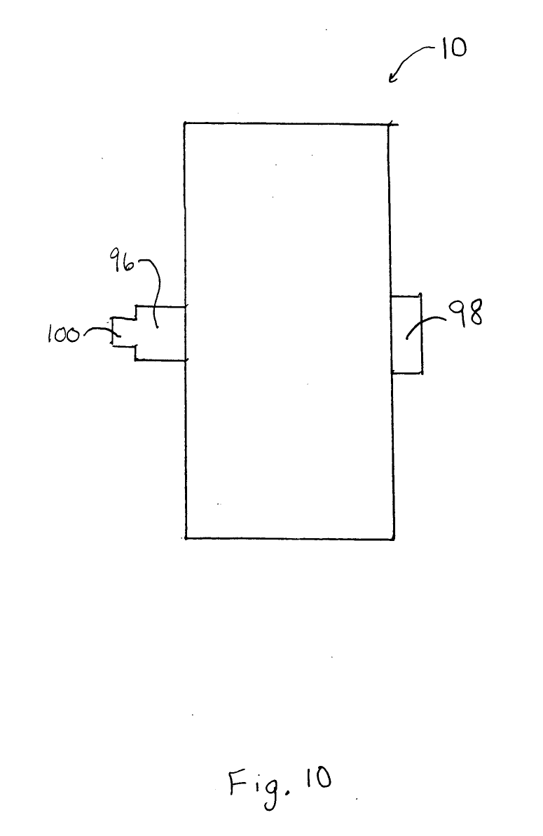

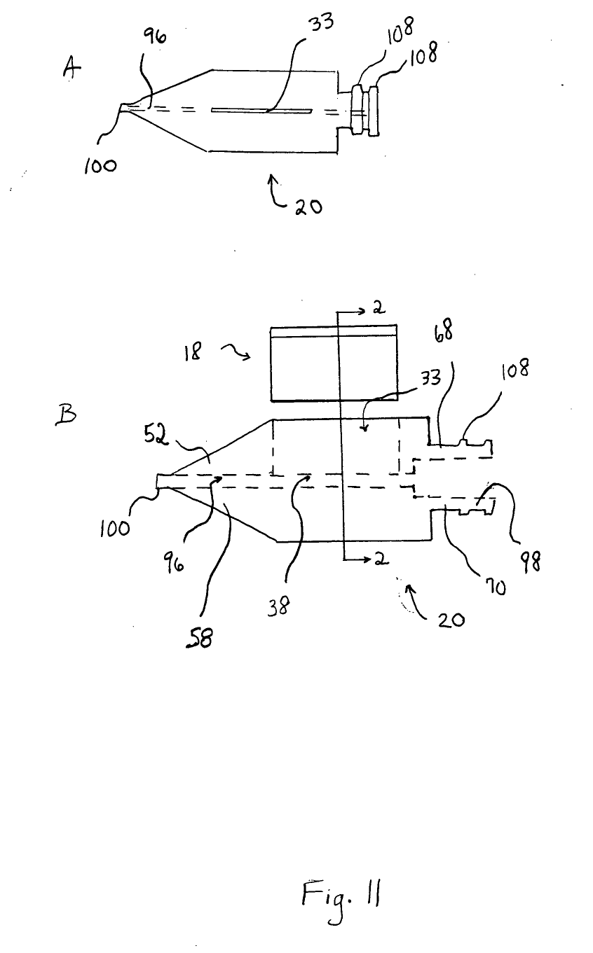

As shown in FIGS. 11A, 11B, 12A, 12B, and 13, in certain embodiments of the lens rolling device 10 the first shelf 24, second shelf 32, and second concave surface 34 are present on the second member 20. As best seen in FIGS. 11A, 11B, and 12B, an opening 33 within the second member 20 is defined by the first shelf 24, second shelf 32, and second concave surface 34. The opening 33 is oriented along the longitudinal axis of the second member 20 and is intended to receive a lens 12 having a diameter of around 11 millimeters, or standard sized lenses 12. In a certain embodiment, the width of the opening 33 is approximately 1.29 millimeters, or in a range from about 1.285 millimeters to about 1.29 millimeters. In such an embodiment, the first member 18 provides only the first concave surface 26 of the cavity 38. The width of the first member 18 which is inserted into the opening 33 of the second member 20 is from about 1.320 millimeters to about 1.325 millimeters. This provides a force fit for the parts. In other embodiments, the first member 18 is lightly larger in width than the width of the opening 33 in order to provide a force fit of the parts. In certain embodiments, the first member 18 may also have a flattened edge 19 to allow a user to comfortably push the first member 18 into engagement with the second member 20.

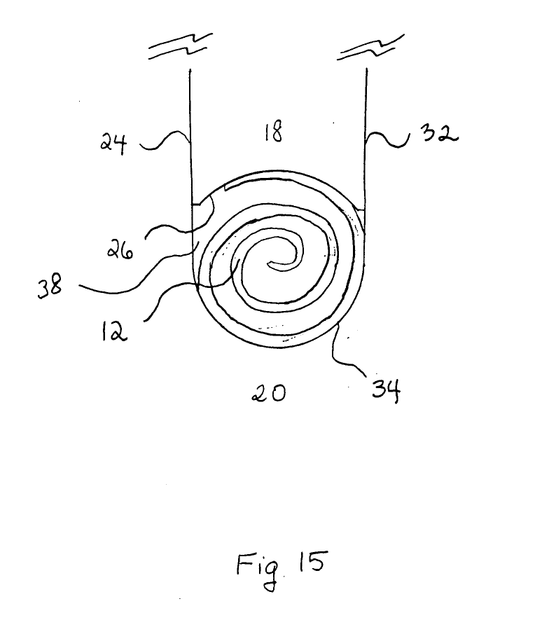

Referring to FIG. 12, the first member 18 is inserted into the second member 20 after placing the lens 12 onto either the first shelf 24 or the second shelf 32 of the second member 20. The size of the cavity 38 is decreased, and the lens 12 is rolled as previously described herein. Note that in certain embodiments the first concave surface 26 has an extended portion 27 which is flattened so that no sharp edge is present which could rip or grasp the lens 12 during the rolling process. In some embodiments, the extended portion 27 has a width of about 5 microns at the point most distal from the body of the first member 18. In alternate embodiments, the first concave surface 26 has a radius surface of 0.79 millimeters and the radius is less than a half circle. Stated another way, if a line were drawn between the two extended portions 27 shown in FIG. 12A, then less than half a circle would be visualized and the shape would more resemble half of an oval. Thus, the first concave surface 26 has a flatter curvature than the second concave surface 34 of the second member. As seen in FIG. 15, the portion of the lens 12 in contact with the more rounded second concave surface 34 initiated rolling and is found in the interior area of the rolled lens 12. The flatter curvature of the first concave surface 26 creates more resistance to movement of the portion of the lens 12 contacting it.

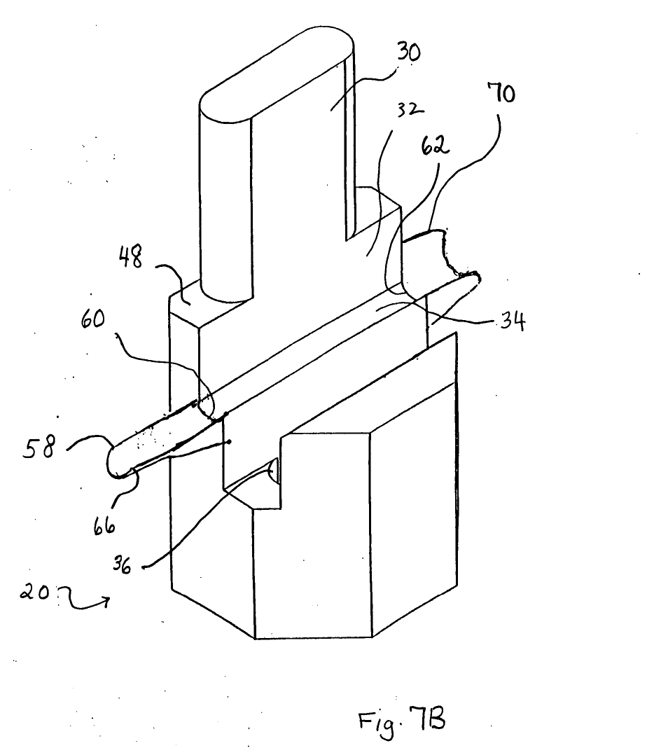

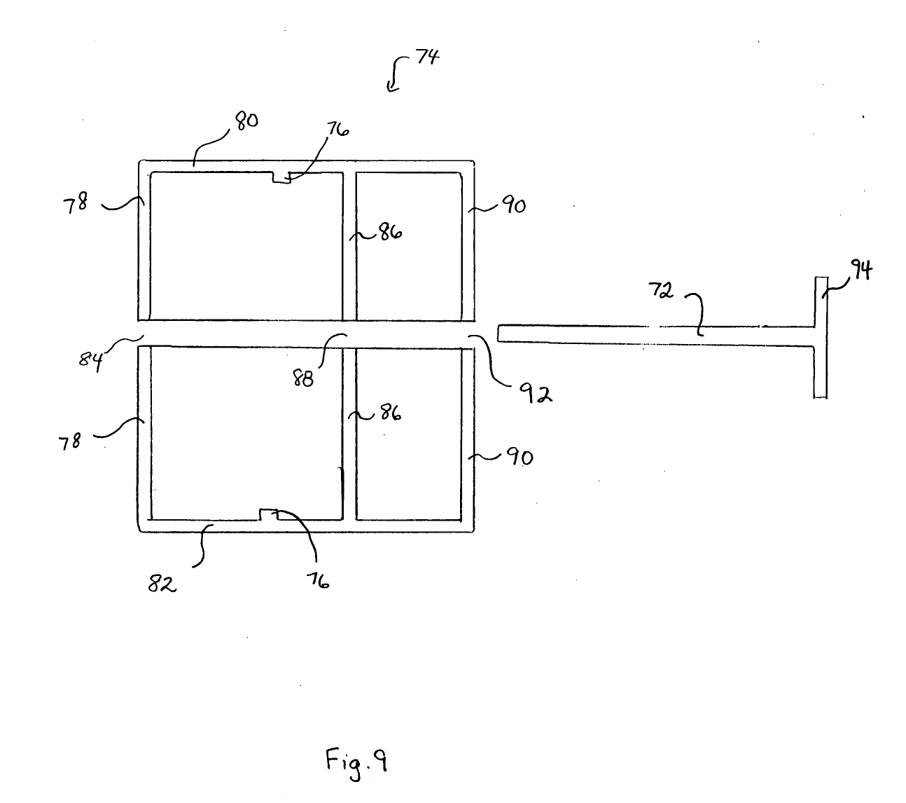

As shown in FIGS. 7A, 7B, and 8, in certain embodiments of the present invention, the lens rolling device 10 further includes a first cannula half 52 attached to the first end 54 of the first concave surface 26 and a second cannula half 58 attached to the first end 60 of the second concave surface 34 of the second member 20. As shown in FIGS. 8 and 10, when the first member 18 fully engages the second member 20 an operational cannula 96 is formed as the first cannula half 52 contacts, or engages, the second cannula half 58. Again, the individual halves are shown in FIG. 8. As shown in FIGS. 7A and 7B, in certain embodiments, the first member 18 and the second member 20 may be identical It is understood that each cannula half may be approximately one half of the cannula, or a portion that is less than or more than one half. Thus the term cannula half is understood to mean a cannula portion, such that two cannula halves, or cannula portions, create an operational cannula upon engagement. The same understanding is present for the funnel halves, which engage to form an operational funnel 98. Accordingly, the funnel halves, or portions, do not have to be exactly one half of the funnel.

The FIGS. 8 and 10 show the operational cannula 96 which has a narrow tip 100 used for inserting the lens 12 through an incision in the cornea of the eye of a patient. The first distal end 64 of the first cannula half 52 and the second distal end 66 of the second cannula half 58 contact in order to form the narrow tip 100 of the operational cannula 96 which is inserted through the incision in the cornea so that the lens 12 is inserted into the eye. The internal diameter of the narrow tip 100 of the operational cannula 96 is from about 1.32 millimeters to about 1.27 millimeters. In alternate embodiments of the present invention, the internal diameter of the operational cannula 96 is from about 1.32 millimeters to about 1.27 millimeters. With regard to the internal diameter of the bore which runs the entire length of the second member 20, as shown in FIGS. 11A and 11B, that internal diameter is from about 1.27 millimeters to about 1.32 millimeters.

There is not a tendency for the lens 12 to hang, tear or become blocked as it moves from the cavity 38 through the operational cannula 96 and into the eye of the patient. As stated above, the operational cannula 96 of the current invention is made when the first cannula half 52 and the second half cannula half 58 are joined. Any seams present from the engagement or connection of those parts is parallel to the axis of movement of the lens 12 when the lens 12 travels from the cavity 38 and into the operational cannula 96. Accordingly, when the lens 12 is pushed from the cavity 38 into the operational cannula 96, the lens 12 will not become stuck or damaged in any capacity. As best seen in FIGS. 8 and 10, the narrow tip 100 of the operational cannula 96 has a thinner wall for insertion of that portion of the operational cannula into an incision site of an eye. The length of the thinner walled section provides a sufficient length for insertion of that portion of the operational cannula into the incision site. The thin walled section of the first distal end 64 of the first cannula half 52 is approximately 20 microns thick. Similarly, the thickness of the thin walled section of the second distal end 66 of the second cannula half 58 is also approximately 20 microns. The length of each of these distal ends is approximately 500 microns, which is approximately the thickness of the cornea. Stated another way, the length of the first distal end 64 of the first cannula half 52 and the length of the second distal end 66 of the second cannula half 58 are about the thickness of the cornea. In alternate embodiments, they are about twice the length of the thickness of the cornea.

As shown in FIGS. 11-13, certain embodiments of the invention have an operational cannula 96 located on the second member 20. As shown in FIG. 11B, the first cannula half 52 and second cannula half 58 are part of the second member 20.

As shown in FIGS. 7, 8 and 10, certain embodiments of the present invention may have an operational funnel 98 in addition to an operational cannula 96. As best seen in FIGS. 7A and 7B, the lens rolling device 10 includes a first funnel half 68 attached to the second end 56 of the first concave surface 26 and a second funnel half 70 attached to the second end 62 of the second concave surface 34 so that the first funnel half 68 and the second funnel half 70 are opposite of the first cannula half 52 and the second cannula half 58. Thus, when the first member 18 engages the second member 20, an operational funnel 98 is provided.

Certain embodiments of the invention, as shown in FIGS. 11-13, have an operational funnel 98 within the second member 20. When the first funnel half 68 and the second funnel half 70 are present on the second member 20 to form the operational funnel 98, the plunger 72 is guided through the device in order to push the lens 12 out of the device and into the eye.

The material of construction for the operational cannula 96 and operational funnel 98 are the same as the materials of construction for the first member 18 and the second member 20 of the lens rolling device 10.

Since each half of the funnel is attached to the respective concave surface in the same manner that the cannula halves are attached to the respective concave surfaces, a seamless transition is provided between the operational funnel 98 and the cavity 38. Stated another way, in a manner similar to the generation of the cavity 38 and the operational cannula 96, the operational funnel 98 if provided upon engagement of the first funnel half 68 and second funnel half 70. With regard to the operational funnel 98, the diameter of the portion closer to the cavity 38 is smaller than the diameter of the operational funnel 98 further from the cavity 38. As further described below, certain embodiments of the present invention include a plunger 72 which the operational funnel 98 guides into the cavity 38 so that the rolled lens 12 is pushed from the cavity 38 through the operational cannula 96 and into the eye of a patient.

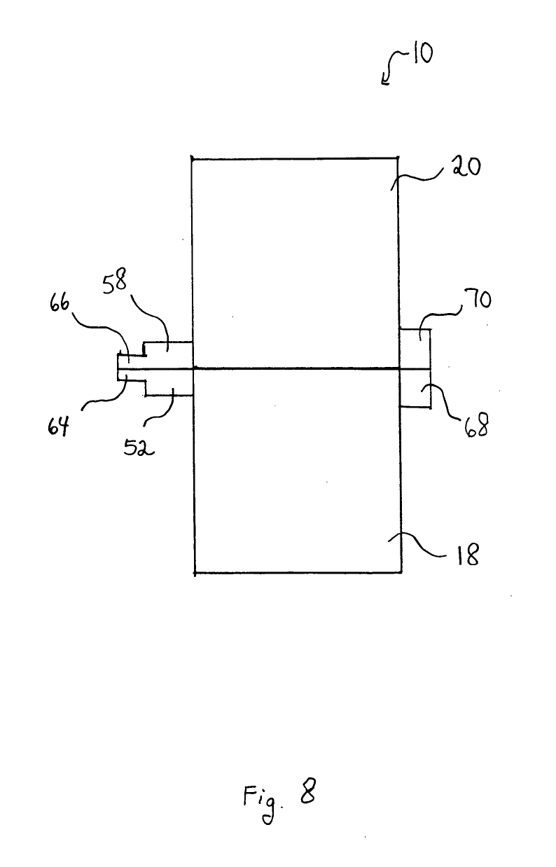

As further described below, an injector barrel 74 may be used to transfer the rolled lens 12 from the cavity 38 into the eye of a patient. An injector barrel 74 is shown in FIG. 9.

As best seen in FIG. 9, an alignment tab 76 holds the lens rolling device 10 in position along an axis perpendicular to the axis of engagement of the first member 18 and the second member 20. The front bulkhead 78 of the injector barrel 74 holds the lens rolling device 10 with the alignment tabs 76. A top horizontal wall 80 and a bottom horizontal wall 82 hold the lens rolling device 10 in a compressed or engaged position. A first hole 84 within the front bulkhead 78 allows the operational cannula 96 to pass therethrough. Also present is a rib 86 which has a first hole 88 to allow insertion of the plunger 72. The back plate 90 of the injector barrel 74 has a first hole 92 located along the center line of the back plate 90. The first hole 92 allows passage therethrough of the plunger 72. Thus, the plunger 72 is positioned by first hole 92 of the back plate 90, and the first hole 88 of the rib 86. Attached to the plunger 72 is a flat plate 94 used to compress the plunger 72 against the rolled lens 12.

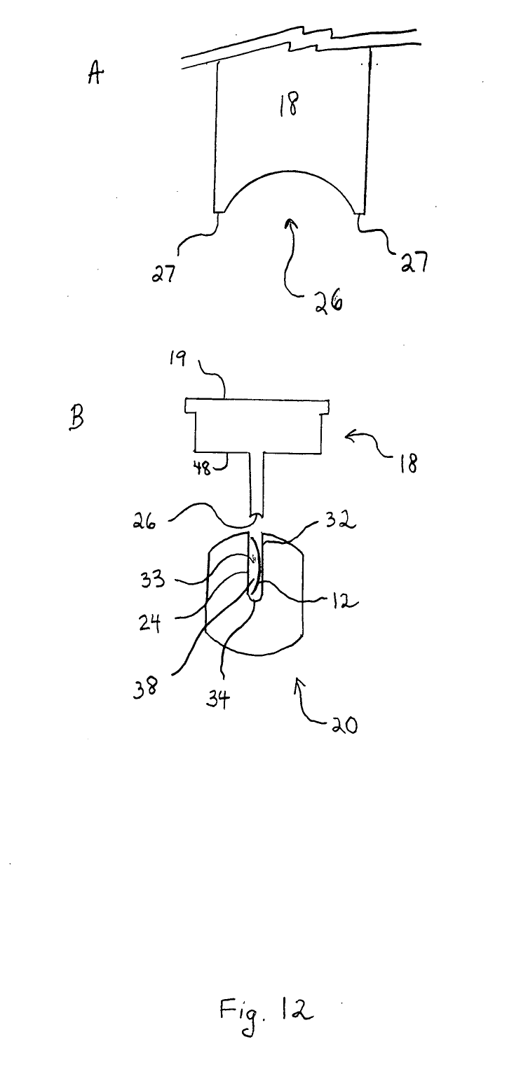

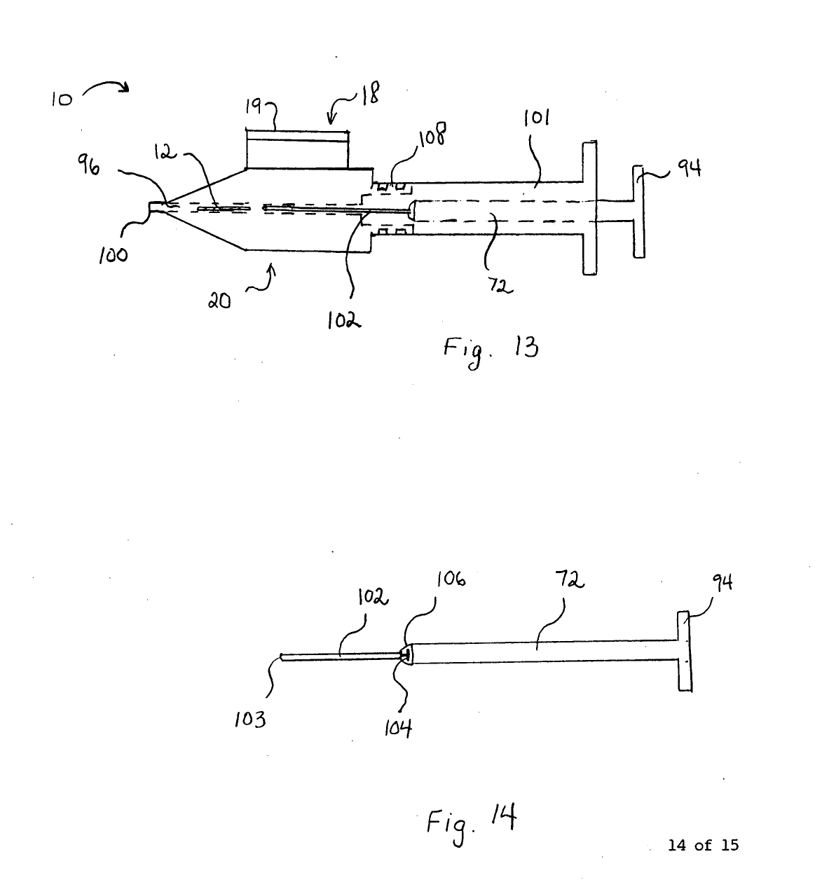

As seen in FIGS. 11-14, certain embodiments of the invention do not use the injector barrel 74 in combination with a plunger 72. As previously described herein, in certain embodiments, a second member 20 contains an operational cannula 96 and operational funnel 98. In such an embodiment, an injector barrel 74 is not required to stabilize the engagement between the first member 18 and the second member 20. As best seen in FIG. 13, the lens rolling device 10 is placed in alignment to receive the plunger 72. Again, the plunger 72 is used to transition the rolled lens 12 from the cavity 38 through the operational cannula 96 and into the eye of a patient.

Still referring to FIG. 13, certain embodiments of the present invention have a push rod 102 which is used to push the rolled lens 12 from the cavity 38 through the operational cannula 96 and into the eye of a patient. The diameter of the push rod 102 is less than the diameter of the cavity 38, operational cannula 96, and operational funnel 98. In certain embodiments, the push rod 102 has a width of approximately 1.27 millimeters. The push rod 102 may be constructed of a rigid material, such as metal or polyethyl-ethyl-ketone (PEEK). The tip 103 of the push rod 102 is slightly rounded in order to avoid unintended ripping or grasping of the lens 12 by a sharp edge of the tip 103. The push rod 102 may be attached to the plunger 72 in a variety of ways. For example, the push rod 102 may have a flange 104 which is embedded into the gasket 106 of the plunger 72, such as a plunger of a commercially available syringe 101. An example of such a syringe is the three millimeter syringe from Becton Dickinson and Company (BD). In certain embodiments, the gasket is removed from the plunger 72 and a hole of approximately 1.3 millimeters is cut in the center thereof. Then the flange 104, is inserted into the gasket. Such an attachment allows the tip 103 of the push rod 102 to be free to self align with the cavity 38 as the push rod 102 moves toward the rolled lens 12.

Methods of Using the Lens Rolling Device

The present invention also disclosed a method of rolling, storing and inserting an intraocular lens into the eye of a patient. The steps of the method include providing a lens rolling device 10, having a first member 18 and a second member 20, placing the lens 12 on a shelf, for example the first shelf 24, and sliding the first member 18 into engagement with the second member 20 so that the lens 12 is rolled and compressed within a cavity 38 formed by the engagement of the first member 18 and second member 20.

If the lens rolling device 10 and the lens 12 are received in a container 14, then the lens 12 is to be removed from the container 14 and the balanced salt solution, or equivalent, within. The lens 12 is placed on the first shelf 24 by using forceps, or an equivalent. More specifically, the lens 12 is placed on the first shelf 24 of the first member 18 so that the convex surface 40 is facing away from the first shelf 24. In certain embodiments of the present invention, as shown in FIG. 6, the first edge 42 of the lens 12 starts to roll inwardly as the second concave surface 34 of the second member 20 moves toward the first concave surface 26 of the first member 18. As the two concave surfaces continue to approach each other, the lens 12 continues to roll up such that the lens is rolled or compressed within the cavity 38. The first edge 42 of the lens 12 being positioned at the interior of the rolled lens 12 and the second edge 44 of the lens 12 having an external location. The direction of rolling is predictable since the lens 12 has a concave/convex shape so that, regardless of the power of the lens 12, it always rolls in the same direction. Also, the unrolling process is predictable.

In alternate embodiments, the lens rolling device 10, with the lens 12 in an unrolled conformation in the cavity 38 thereof, is removed from a bottle of balanced salt solution, and squeezed to roll the lens 12.

Subsequent to compressing, or rolling, of the lens, the lens rolling device 10 may be place in the container 14 which contains a balanced salt solution, or equivalent. The rolled lens 12 and lens rolling device 10 are place within the container 14 for storage purposes. Under such conditions, the rolled lens 12 may be stored for up to thirty minutes.

At the time for insertion of the lens 12 into the eye of a patient, the lens 12 may be removed from the cavity 38 as disclosed below. In certain embodiments of the present invention, the surgeon may disengage the first member 18 from the second member 20 in order to expose the rolled lens 12. The surgeon may then grasp the rolled lens 12 with forceps, or an equivalent, and place the rolled lens 12 into the eye of the patient. Such a transitioning of the rolled lens 12 may be accomplished using a lens rolling device 10 which does not have an operational cannula 96 or an operational funnel 98. Also, the method may be performed using a lens rolling device 10 which does have an operational cannula 96 and an operational funnel 98.

In an alternate embodiment of the present invention, when a lens rolling device 10 which has an operational cannula 96 and an operational funnel 98 is used, after the lens 12 is rolled as described above, the rolled lens 12 may be pushed from the cavity 38 into the operational cannula 96 and ultimately into the eye of the patient. At the time for insertion of the lens 12, the lens rolling device 10 is placed in the injector barrel 74 such that the operational cannula 96 is received by the first hole 84 of the front bulkhead 78. The injector barrel 74 holds the first member 18 and second member 20 in a fully engaged position. The plunger 72 is positioned to be received by the first hole 92 of the back plate 90 and the first hole 88 of the rib 86, so that the plunger 72 is received in the cavity 38. The plunger 72 is then pushed through the cavity 38 so that the lens 12 is expelled from the cavity 38. The lens 12 travels from the cavity 38 through the operational cannula 96 and into the eye of the patient. Accordingly, the lens 12 is inserted into the eye through an incision in the cornea.

In another embodiment, the rolled lens 12 is discharged from the lens rolling device 10 without the use of the injector barrel 74. In the same manner, a plunger 72 is used to push the rolled lens 12 from the cavity 38 through the operational cannula 96 and into the eye. Certain embodiments may additionally use a push rod 102 in order to push the rolled lens 12 from the cavity 38 through the operational cannula 96 and into the eye of the patient. A syringe having a plunger 72 may be attached to the second member 20 by a standard luer lock 108, in the same manner that a needle and syringe engage. In still other embodiments, a viscoelastic material, for example Healon® by Pharmacia, is injected into the second member 20 and an unrolled lens 12 is placed on either the first shelf 24 or second shelf 32 of the second member 20 and rolled by engaging the first member 18 and the second member 20, as described herein.

In other embodiments of the present invention, after the lens 12 has been rolled, the narrow tip 100 of the operational cannula 96 is placed in the corneal incision which has a size of one millimeter or less. The plunger 72 is pushed into the cavity 38 and the rolled lens 12 is injected into the eye through the operational cannula 96. As best seen in Figurer 13 and 14, in other embodiments, a syringe 101 is attached to the second member 20, before or after rolling the lens 12, and the lens 12 is pushed from the cavity 38 through the operational cannula 96 and into the incision by a rod 102 which is attached to the plunger 72 of a syringe 101. The plunger 72 and rod 102 are easily manipulated by the hands of the user. The thin lens 12 is designed to unroll within the eye in approximately 15 seconds after coming in contact with the warm aqueous of the eye. The narrow tip 100 of the operational cannula 96 is removed from the incision site and the surgeon positions the lens 12 using the same incision opening. In certain embodiments of the present invention, the incision in the cornea is from about 2 millimeters to about 0.5 millimeters. In still other embodiments of the present invention, the incision in the cornea is from about 1 millimeter to about 0.25 millimeters.

In all of the embodiments of the present invention, it is understood that the lens 12 is placed on the shelf of a member of the lens rolling device 10. It is understood that the lens 12 may be place upon either the first shelf 24 of the first member 18, or the second shelf 32 of the second member 20. Upon engagement of the first member 18 and the second member 20, the lens 12 is placed, or positioned, between two shelves, specifically the first shelf 24 and the second shelf 32.

All references, publications, and patents disclosed herein are expressly incorporated by reference.

Thus, it is seen that the apparatus and method of the present invention readily achieves the ends and advantages mentioned as well as those inherent therein. While certain preferred embodiments of the invention have been illustrated and described for purposes of the present disclosure, numerous changes in the arrangement and construction of parts may be made by those skilled in the art, which changes are encompassed within the scope and spirit of the present invention as defined by the following claims.

1. A lens rolling system, comprising:
a first member having a first concave surface; and
a second member having an opening defined by a first shelf, a second shelf, and a second concave surface, wherein the first member removably engages the opening of the second member, so that a cavity is formed.

2. The lens rolling system of claim 1, further comprising the second member having an operational cannula, wherein the operational cannula aligns with the cavity so that a lens located in the cavity may exit the cavity through the operational cannula.

3. The lens rolling system of claim 2, further comprising the second member having an operational funnel, wherein the operational funnel aligns with the cavity so that the operational cannula, the cavity, and operational funnel define an opening therethrough.

4. The lens rolling system of claim 3, further comprising a rod removably engaged to the operational funnel, cavity, and operational cannula.

5. The lens rolling system of claim 3, further comprising a luer lock attached to the second member.

6. A method of rolling an intraocular lens, comprising:
providing a first member having a first concave surface;
providing a second member having an opening defined by a first shelf, a second shelf, and a second concave surface;
inserting a lens into the opening of the second member;
engaging the first member with the second member so that the lens rolls upon itself, and
moving the lens from the engaged first member and second member into an eye.

7. The method of claim 6, wherein moving the lens further comprises pushing the lens through an operational cannula.

8. The method of claim 7, further comprising attaching a syringe to the second member.

9. The method of claim 7, further comprising irrigating the lens with a fluid.

10. The method of claim 6, wherein moving the lens further comprises contacting the lens with a rod and advancing the lens through an operational cannula and out of the engaged first member and second member.

11. The method of claim 10, further comprising placing a tip of the operational cannula into an incision of the eye.

12. An apparatus for rolling, storing, and inserting an intraocular lens, comprising:
a first member having a first arm, a first cannula half, a first funnel half, a first surface, a second surface, and the first member having a first hole defined therein; and
a second member having a second arm, a second cannula half, a second funnel half, and the second member having a second hole defined therein.

13. The apparatus of claim 12, further comprising a plunger slidably engaged with the first funnel half and the second funnel half.

14. A method of rolling an intraocular lens, comprising:
providing a lens rolling device, having a first member and a second member;
placing a lens on a shelf of the first member; and
sliding the first member into engagement with the second member so that the lens is rolled and compressed within a cavity formed by the engagement of the first member and second member.

15. The method of claim 14, further comprising:
positioning a plunger in the cavity; and
pushing the plunger through the cavity so that the lens is expelled from the cavity.

16. The method of claim 15, wherein placing the lens on the shelf further comprises placing the lens between two shelves.

17. The method of claim 15, further comprising inserting the lens into an eye through an incision in a cornea.

18. The method of claim 17, wherein the incision in the cornea is from about 2 millimeters to about 0.5 millimeter.

19. The method of claim 18, wherein the incision in the cornea is from about one millimeter to about 0.25 millimeters.

20. A lens rolling device, comprising:
a first member having a first insertion arm, a first shelf, a first concave surface, the first concave surface having a first end and a second end, and the first member having a first hole defined therein; and
a second member having a second insertion arm, a second shelf, a second concave surface, the second concave surface having a first end and a second end, and the second member having a second hole defined therein.

21. The device of claim 20, wherein a cavity is formed when the first concave surface and the second concave surface engage.

22. The device of claim 21 wherein the cavity is round.

23. The device of claim 21 wherein the cavity is oval

24. The device of claim 20 further comprising a first cannula half attached to the first end of the first concave surface and a second cannula half attached to the first end of the second concave surface of the second member, wherein the first cannula half has a first distal end and the second cannula half has a second distal end.

25. The device of claim 24, wherein a diameter of the first distal end of the first cannula half and a diameter of the second distal end of the second cannula half are less than about 1.32 millimeters.

26. The device of claim 24 wherein a length of the first distal end of the first cannula half and a length of the second distal end of the second cannula half are about a thickness of a cornea.

27. The device of claim 24 further comprising a first funnel half attached to the second end of the first concave surface and a second funnel half attached to the second end of the second concave surface so that the first funnel half and the second funnel half are opposite of the first cannula half and second cannula half.

28. The device of claim 27 further comprising a container so that the device is sealed and sterilized in the container.

29. The device of claim 28 further comprising a plunger frictionally engaged with the cavity so that the first funnel half and the second funnel half align the plunger and the cavity.
